(19) United States
(12) Patent Application Publication
(10) Pub. No.: US 2015/0309010 A1
(43) Pub. Date: Oct. 29, 2015

(54) BIOLOGICAL INFORMATION MEASURING DEVICE, BIOLOGICAL INFORMATION MEASURING SYSTEM USING SAME BIOLOGICAL INFORMATION MEASURING DEVICE, AND METHOD USING SAME BIOLOGICAL INFORMATION MEASURING DEVICE

(71) Applicant: Panasonic Healthcare Holdings Co., Ltd., Minato-ku, Tokyo (JP)

(72) Inventor: Tatsuhiko Furukawa, Ehime (JP)

(21) Appl. No.: 14/649,550

(22) PCT Filed: Dec. 9, 2013
(86) PCT No.: PCT/JP2013/007231

§ 371 Date: Jun. 4, 2015

(30) Foreign Application Priority Data
Dec. 28, 2012 (JP) 2012-287109
Jul. 12, 2013 (JP) 2013-146133

(51) Int. Cl. G01N 33/487; G01N 33/49

(52) U.S. Cl. CPC G01N 33/48785; G01N 33/49

(57) ABSTRACT

A biological information measuring device comprises a sensor mounting portion that is provided to a main case and is used for mounting a sensor that measures biological information, a measurement component that is connected to the sensor mounting portion, a controller that is connected to the measurement component, a memory that is connected to the controller, and a timer. The controller associates measurement variance cause information acquired in a single measurement of biological information by the measurement component with the measurement results obtained by this single measurement, and stores the information in the memory. Accordingly, the user of the biological information measuring device can be prompted to use it properly.

### PRIORITY

This is a National Stage Application of International Application PCT/JP2013/007231, with an international filing date of Dec. 9, 2013, which claims priority to Japanese Patent Application No. 2012-287109 filed on Dec. 28, 2012 and Japanese Patent Application No. 2013-146133 filed on Jul. 12, 2013. The entire disclosures of International Application PCT/JP2013/007231, Japanese Patent Application No. 2012-287109 and Japanese Patent Application No. 2013-146133 are hereby incorporated herein by reference.

### TECHNICAL FIELD

Embodiments of the present invention relate to a biological information measuring device that measures biological information, such as a blood glucose value, from blood or another such biological sample, and to a biological information measurement system, etc., in which this device is used.

### BACKGROUND

A conventional biological information measuring device of this type (hereinafter referred to as measurement device) comprised, for example, a main case having a sensor mounting portion for mounting a biological information measurement sensor (hereinafter referred to as sensor), a measurement component that was connected to this sensor mounting portion, a controller that was connected to this measurement component, a memory that was connected to this controller, and a timer. Measured values produced by the measurement component were sent to the personal computer of a physician, and the physician utilized them as data in giving health consultations (see Patent Literature 1 below, for example).

It is well known that the measured values produced by this type of measurement device fall within a range of permissible error (ISO 15197 (2003), for example, states that “at a blood glucose value of less than 75 mg/dL, at least 95% of the measured values shall be within ±15 mg/dL”).

Patent Literature 1: Japanese Laid-Open Patent Application 2012-230521

### SUMMARY

In general, variance occurs in measured values, depending on how a patient uses a measurement device, even though the variance may be within the above-mentioned range of permissible error. For instance, variance within the range of permissible error may occur in measured values depending on the ambient temperature during measurement and on how much time passes between the mounting of the sensor to the sensor mounting portion and the actual measurement.

However, in the conventional example given above, since only the measured values are sent to a physician, the physician and the patient cannot ascertain the usage state of the measurement device with which these measured values were found. Therefore, the physician or patient cannot be prompted to use the measurement device properly even if variance in the measured values should occur due to how the measurement device was used.

In view of this, it is an object of certain embodiments of the present invention to be able to prompt a user to use a measurement device in a more appropriate way.

According to a first aspect of the present invention, a biological information measuring device comprises a main case, a sensor mounting portion that is provided to the main case and is used to mount a sensor for measuring biological information, a measurement component that is connected to the sensor mounting portion, a controller that is connected to the measurement component, a memory that is connected to the controller, and a timer. The controller associates measurement variance cause information acquired in a single measurement of biological information by the measurement component with the measurement results obtained by this single measurement, and stores the information in the memory.

According to a second aspect of the present invention, a method, in which is used a biological information measuring device for measuring biological information with a sensor, comprises the steps of acquiring, in the measurement of biological information, measurement variance cause information that includes one or more of information about the temperature during measurement of the biological information, information about the duration from when the sensor is mounted to the sensor mounting portion until the measurement component measures the biological information, and information about the quantity of biological sample deposited on the sensor, producing measurement improvement data indicating whether or not the measurement variance cause information is outside the reference values, every time the biological information is measured, storing the produced measurement improvement data in a memory, and outputting an analysis result based on the measurement improvement data stored in the memory to a display component.

When a measurement is taken, the controller of the present invention acquires measurement variance cause information during the measurement in order to encourage more appropriate use of the measurement device, associates this measurement variance cause information with the current measured value produced by the measurement component, and stores this information in a memory.

Therefore, when the measured values stored in the memory of the measurement device are sent to the personal computer of a physician along with measurement variance cause information, for example, that physician can be apprised of the usage state of the measurement device based on the measurement variance cause information displayed on his personal computer, and can advise the patient of a more appropriate way to use the measurement device.

As a result, this is an effective way to encourage users to use a measurement device more appropriately.

### BRIEF DESCRIPTION OF THE DRAWINGS

FIG. 1 is a simplified diagram of a biological information measurement system that makes use of the biological information measuring device pertaining to Embodiment 1;

FIG. 2 is a diagram of the configuration of this biological information measuring device;

FIGS. 3A and 3B show recorded data in this biological information measuring device;

FIG. 4 is a flowchart of the operation of this biological information measuring device;

FIG. 5 shows the configuration of a data processing device in this biological information measuring device;

FIG. 6 is a flowchart of the operation of the data processing device in this biological information measuring device;

FIG. 7 is a flowchart of the operation of the data processing device in this biological information measuring device;

FIG. 8 is a flowchart of the operation of the data processing device in this biological information measuring device;

FIG. 9 shows a screenshot from the display component of the data processing device in this biological information measuring device;

FIG. 10 shows a screenshot from the display component of the data processing device in this biological information measuring device;

FIG. 11 shows a screenshot from the display component of the data processing device in this biological information measuring device;

FIG. 12 shows a screenshot from the display component of the data processing device in this biological information measuring device;

FIG. 13 shows a screenshot from the display component of the data processing device in this biological information measuring device;

FIG. 14 shows a screenshot from the display component of the data processing device in this biological information measuring device;

FIG. 15 shows a screenshot from the display component of the data processing device in this biological information measuring device;

FIG. 16 shows a screenshot from the display component of the data processing device in this biological information measuring device;

FIG. 17 is a flowchart of the operation of the biological information measuring device pertaining to Embodiment 2;

FIG. 18 is a front view of the display component of this biological information measuring device;

FIG. 19 is a front view of the display component of this biological information measuring device;

FIGS. 20A to 20E are front views of the display component of this biological information measuring device; and

FIG. 21 shows an example of recorded data in this biological information measuring device.

### DETAILED DESCRIPTION

Embodiments will now be described in detail through reference to the drawings as needed. However, some unnecessarily detailed description may be omitted. For example, detailed description of already known facts or redundant description of components that are substantially the same may be omitted. This is to avoid unnecessary repetition in the following description, and facilitate an understanding on the part of a person skilled in the art.

The inventors have provided the appended drawings and the following description so that a person skilled in the art might fully understand this disclosure, but do not intend for these to limit what is discussed in the patent claims.

An embodiment of the present invention will now be described through reference to the appended drawings.

### 1-1 Biological Information Measurement System 100

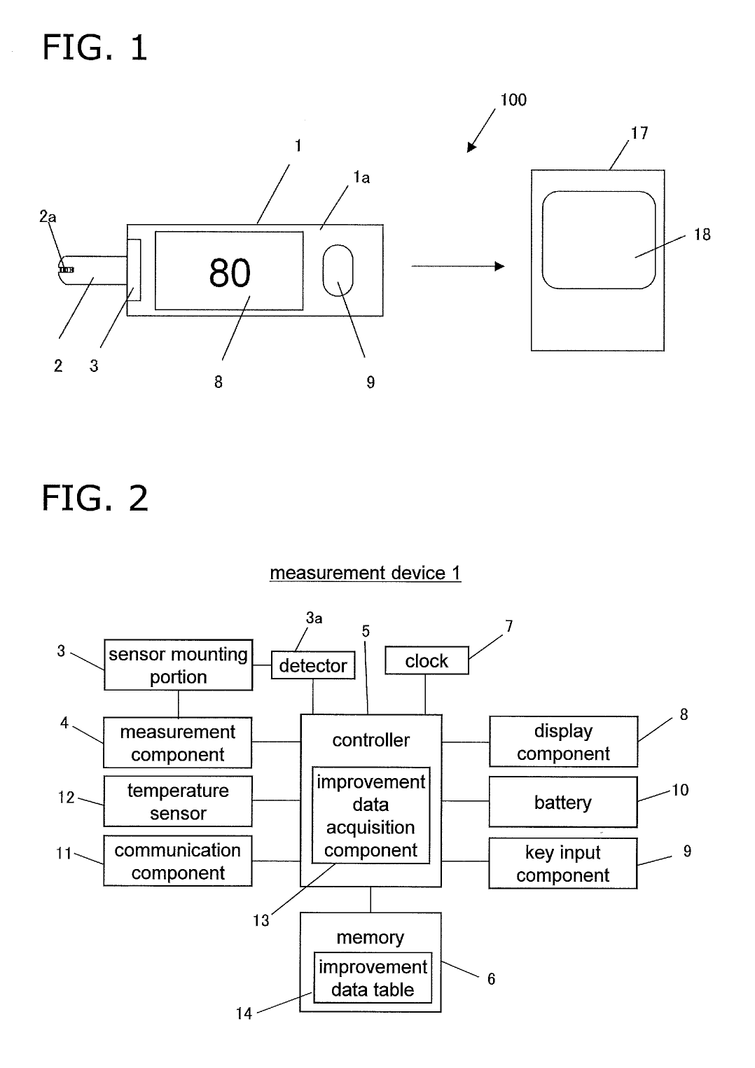

FIG. 1 is a simplified diagram of a biological information measurement system 100 pertaining to this embodiment. The biological information measurement system 100 comprises a measurement device 1 (an example of a measurement device) and an analyzer 17 (an example of a data processing device) capable of communicating with the measurement device 1. The measurement device 1 is used to measure biological information such as blood glucose values, for example. In the following example, the measurement device 1 measures a blood glucose value (an example of biological information) when blood (an example of a biological sample) is deposited on a biosensor. The analyzer 17 is a device used by a physician or other person who analyzes measured values, and is, for example, a PC, a smart phone, a tablet terminal or other such portable terminal device, or another such apparatus.

The measurement device 1 and the analyzer 17 communicate via a USB cable or other such wired system, or by near field wireless communication.

### 1-2 Measurement Device 1

### 1-2-1 Configuration of Measurement Device 1

As shown in FIG. 1, the measurement device 1 has a rectangular main case 1a. This main case 1a comprises a sensor mounting portion 3 (an example of a sensor mounting portion) to which a sensor 2 (an example of a sensor) is mounted.

As shown in FIG. 2, the main case 1a comprises in its interior a measurement component 4 (an example of a measurement component) that is connected to the sensor mounting portion 3, a controller 5 (an example of a controller) that is connected to this measurement component 4, a memory 6 (an example of a memory) that is connected to this controller 5, and a clock 7 (an example of a timer). The controller 5 is connected to a display component 8 and a key input component 9 (which are also shown in FIG. 1), as well as a rechargeable battery 10, a communication component 11, and a temperature sensor 12. There is also provided a detector 3a that is connected to the sensor mounting portion 3, detects when the sensor 2 is mounted or removed, and notifies the controller 5 to this effect. The controller 5 is constituted by a CPU or other such processor that executes various functions according to specific programs. A display component 18 is constituted by an LCD, an organic EL display, or the like.

When this measurement device 1 is used to make a measurement, as is well known, there may be variance in the measured value, within a range of permissible error, depending on how the patient uses the measurement device 1, that is, depending on the measurement skill of the patient. In view of this, the controller 5 in this embodiment acquires measurement variance cause information as information related to possible causes for the occurrence of measurement variance due to the measurement skill of users, in order to improve the measurement skill of the patient. More specifically, an improvement data acquisition component 13 in the controller 5 in FIG. 2 acquires measurement variance cause information for every measurement.

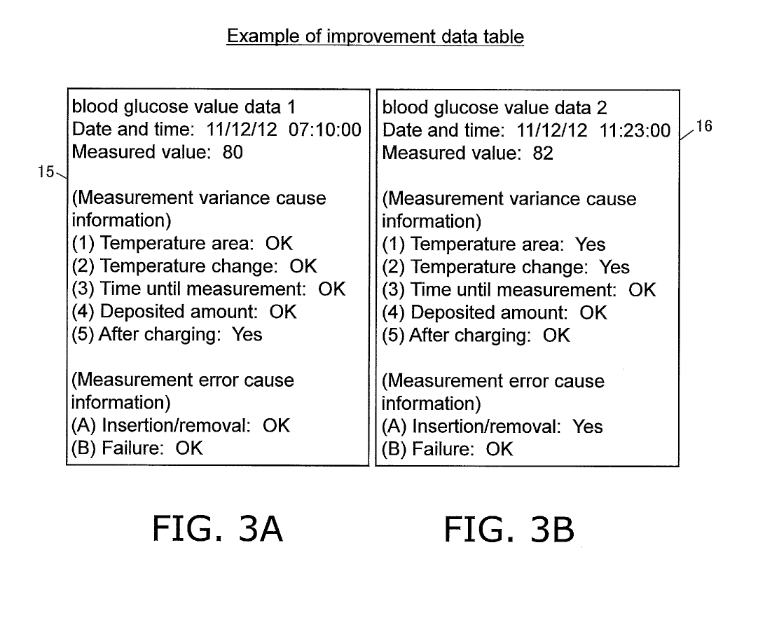

The controller 5 produces data in which measured values and the date and time when measurement is executed by the measurement component 4 (an example of measurement results) are associated with measurement variance cause information acquired by the controller 5 when these measured values were acquired, and stores this data in the memory 6. More specifically, the controller 5 associates the current measured value obtained by the measurement component 4 with measurement variance cause information acquired by the controller 5 during the current measurement, and produces the measurement improvement data 15 and 16 shown in FIGS. 3A and 3B. This measurement improvement data 15 and 16 is stored in an improvement data table 14 in the memory 6.

### 1-2-2 Production of Measurement Improvement Data

Measurement variance cause information and measurement improvement data that holds this measurement variance cause information will be described using the measurement improvement data 15 in FIG. 3A and the measurement improvement data 16 in FIG. 3B as examples.

The measurement improvement data 15 and 16 include two types of information: “measurement variance cause information” and “measurement error cause information.” The measurement variance cause information is information related to possible causes for the occurrence of measurement variance due to the measurement skill of the user, and has five points of cause data (discussed below), for example. The measurement error cause information is information related to measurement errors due to the measurement skill of the user, and has two points of cause data (discussed below), for example.

These seven points of cause data (the five points of measurement variance cause information and the two points of measurement error cause information) are acquired by the improvement data acquisition component 13 of the controller 5 for every measurement. The category to be improved when the user is taking a measurement is determined by the seven points of cause data.

First, the measurement variance cause information will be described. This measurement variance cause information is made up of the following five points of cause data, for example.

(1) Temperature Area

The temperature area is information (an example of temperature information) indicating whether or not measurement was performed in a specific temperature range (such as between 10 and 40 degrees) from 25 degrees (the recommended measurement temperature). Measurement is preferably performed under a 25-degree environment, which is the recommended measurement temperature. For example, if measurement is performed outdoors, measurement may not fail, but it may be outside a specific temperature range. The reference value for this temperature area is between 10 and 40 degrees, for example. The recommended measurement temperature is 25 degrees, for example.

(2) Temperature Change

A temperature change is information indicating whether or not there has been a temperature change of at least a specific value (such as at least 1 degree/minute) from the actuation of the measurement device 1 until measurement. Measurement is preferably performed in a state in which the internal temperature of the measurement device 1 is the same as the temperature in the measurement environment. For instance, when the measurement device 1 is actuated by mounting the sensor 2 to the sensor mounting portion 3 indoors, and then the measurement device 1 is carried outdoors before a blood glucose value is measured, it is possible that there will be a temperature change of over the specified value between the time of actuation and the measurement. The reference value for this temperature change is 1 degree/minute, for example.

(3) Time Until Measurement

The time until measurement is information (an example of time information after sensor mounting) indicating whether or not a time of at least a reference value (such as 10 minutes) has elapsed between the actuation of the measurement device 1 and measurement. There is the risk that measurement performance will suffer if the sensor 2 is left exposed to the air. Thus, measurement is preferably performed right away. For example, if the user mounts the sensor 2 to the sensor mounting portion 3 and then becomes distracted by watching television, more time than the reference value may end up passing between actuation and measurement. The reference value for this time until measurement is 10 minutes, for example.

(4) Deposited Amount

The deposited amount is information (an example of information about the amount of biological sample) indicating whether or not any additional blood was added during measurement. The measurement is preferably performed without any additional blood being used. The reference value for the deposited amount is that additional blood was used.

(5) After Charging

The time after charging is information (an example of information about the time after charging) indicating whether or not the measurement device 1 was actuated after a reference value of time (such as 10 minutes) had elapsed since the charging of the battery 10 was complete. The measurement is preferably performed in a state in which charging has not raised the temperature inside the measurement device 1. For example, if the user forgets to charge the measurement device 1 and then hurriedly charges it and performs measurement, this results in a situation in which the measurement ends up being taken within the reference value of time since the completion of charging. The reference value for after charging is 10 minutes, for example.

Next, the measurement error cause information will be described. The measurement error cause information is made up of the following two points of cause data, for example.

(A) Insertion/Removal

Insertion/removal is information (an example of sensor insertion/removal information) indicating how many times the sensor 2 has been inserted into and removed from the sensor mounting portion 3. For example, if the sensor 2 is mounted backward to the sensor mounting portion 3, it has to be taken out and remounted to the sensor mounting portion 3 in the proper orientation. When this happens, since the user's fingers touch the sensor 2 a number of times, there is the risk of soiling, transfer of body temperature, or other such problems, and these should be avoided as much as possible. The reference value for this insertion/removal is that the sensor 2 has been inserted or removed.

(B) Failure

Failure is information (measurement failure information) indicating whether or not measurement failure occurred during any measurement prior to the current normal measurement. For example, measurement may fail due to abnormal ambient temperature (such as measurement at below 10 degrees), impact during measurement (when the measurement device 1 is dropped), or some other such reason. The reference value for this failure is that there was a failure.

These seven points of cause data are associated with the measured values and the date and time when measurement was performed, thus producing the measurement improvement data 15 and 16. The measurement improvement data 15 and 16 are recorded to the improvement data table 14 of the memory 6.

The various reference values for the cause data of the measurement variance cause information and the cause data of the measurement error cause information are stored ahead of time in the memory 6. These reference values are read out (acquired) by the controller 5, and the controller 5 stores the result of determining whether or not values are outside the reference values as the measurement improvement data 15 and 16.

### 1-2-3 Operation of Measurement Device 1

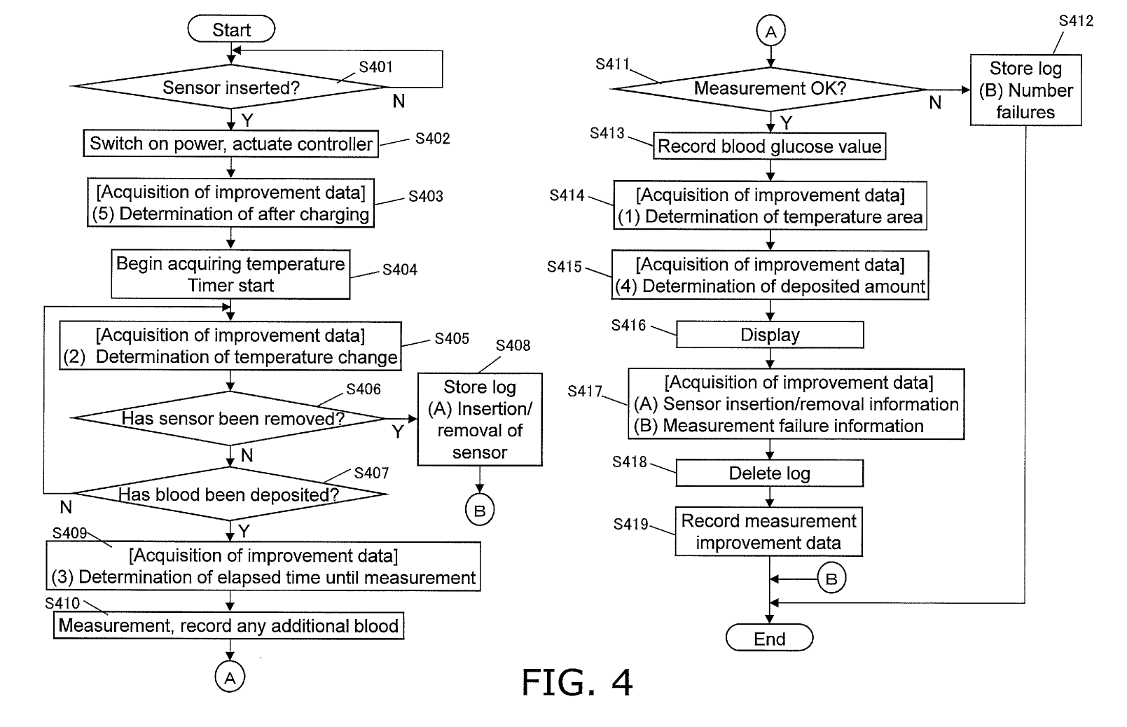

The processing to produce measurement improvement data and the acquisition of measured values will now be described through reference to FIG. 4. The process of producing the measurement improvement data 15 in FIG. 3A will be described as an example.

When the user mounts the sensor 2 in FIG. 1 to the sensor mounting portion 3 of the measurement device 1 (S401), the power is switched on and the controller 5 of the measurement device 1 is actuated (S402).

After this, the controller 5 starts measuring the blood glucose value and acquiring improvement data.

First, the “(5) After Charging” information, which is one of the cause data of the improvement data, is checked by acquiring information about the time after charging with the improvement data acquisition component 13 of the controller 5 (S403). More specifically, the improvement data acquisition component 13 of the controller 5 uses the current time on the clock 7 and the time at which the charging of the battery 10 was last finished (stored in the memory 6) to calculate the elapsed time since the completion of charging. it is then determined whether or not the measurement device 1 was actuated more than a specific amount of time (such as 10 minutes) since the completion of charging of the battery 10. This specific length of time is stored ahead of time in the memory 6 as mentioned above, as a reference value. If the measurement device 1 was actuated less than the specific length of time since the completion of charging of the battery 10, the improvement data acquisition component 13 determines that there is room for improvement in the measurement, and records this to the improvement data table 14. In this case, it is recorded as “Yes” to (5) After Charging, as shown in the measurement improvement data 15 in FIG. 3A.

Next, the controller 5 changes to a mode of sensing the temperature near the sensor mounting portion 3 at specific intervals (S404). At this point, the controller 5 uses the temperature sensor 12 to continuously sense the temperature near the sensor mounting portion 3 at specific intervals, according to the timer function of the clock 7, until blood is deposited on a deposition portion 2a. The temperature sensed at these specific intervals is used to repeatedly calculate the temperature change over a specific length of time.

The improvement data acquisition component 13 of the controller 5 then checks the information for “(2) Temperature Change” (S405). More specifically, the improvement data acquisition component 13 determines that there is room for improvement in the measurement if the temperature change over the specific length of time is above a reference value (such as 1 degree/minute), and records this to the improvement data table 14. In the current measurement, let us assume that there was no temperature change above the specific value up until blood was deposited on the deposition portion 2a. In this case, as shown in FIG. 3A, the (2) Temperature Change of the measurement variance cause information is recorded as “OK.”

Also, the controller 5 detects that the sensor 2 has been removed from the sensor mounting portion 3 up until blood is deposited on the deposition portion 2a (S406 and S407). Steps S405 to S407 are repeated until blood is deposited on the deposition portion 2a.

In S406 in FIG. 4, when the sensor 2 is removed from the sensor mounting portion 3, the fact that the sensor 2 has been inserted or removed is stored by the improvement data acquisition component 13 in the memory 6 as a sensor insertion/removal log (S408). After this, the controller 5 ends the measurement operation. When the sensor 2 has thus been removed, and measurement is therefore unsuccessful and no measured value can be acquired, this sensor insertion/removal log is added as measurement improvement data to the measured value acquired the next time a measurement is successful.

Next, upon notification of the deposition of blood by the measurement component 4, the controller 5 acquires the next cause data, that is, information about the elapsed time until measurement, and checks the “(3) Time Until Measurement” (S409). More specifically, the improvement data acquisition component 13 uses the clock 7 to calculate the elapsed time from when the measurement device 1 is actuated until blood is deposited. If a length of time over the reference value until measurement (such as 10 minutes) has elapsed, it is determined that there is room for improvement in the measurement, and this is recorded to the improvement data table 14. In the current measurement, we will assume that a length of time over the reference value until measurement has not elapsed. In this case, as shown in FIG. 3A, the (3) Time Until Measurement of the measurement variance cause information is recorded as “OK.”

After this, the blood glucose value is measured by the measurement component 4 (S410). During this blood glucose value measurement, if any additional blood is added, the measurement component 4 makes a log entry of additional blood “added” in the memory 6.

If the measurement fails, such as when measurement is performed outside the measurement reference temperature range (such as 5 to 45 degrees) (S411), the improvement data acquisition component 13 makes a log entry of measurement “failed” in the memory 6 (S412). After this, the controller 5 ends the measurement operation. That is, when measurement does not succeed, and no measured value can be acquired, measurement failure log is added as measurement improvement data to the measured value acquired the next time a measurement is successful.

If measurement is carried out normally, the controller 5 records the measured blood glucose value in the memory 6, and the improvement data acquisition component 13 records the measurement date and time, along with the blood glucose value, to the measurement improvement data 15 in FIG. 3A (S413). For instance, this measurement improvement data 15 indicates data measured at 7:10 and 00 seconds a.m. on the morning of Nov. 12, 2012, and the blood glucose value at that time was 80.

The controller 5 then checks the “(1) Temperature Area” information (S414). More specifically, the improvement data acquisition component 13 acquires the current measurement temperature from the temperature sensor 12, and checks whether or not it is within a reference value (such as 10 to 40 degrees). If the measurement temperature is outside the reference value, it is determined that there is room for improvement in the measurement, and this is recorded to the improvement data table 14. The reference value (such as 10 to 40 degrees) used to determine this temperature area is a narrower temperature range than the reference value temperature range (such as 5 to 45 degrees) used to determine a measurement error, and is included in the reference value temperature range used to determine a measurement error. Therefore, the cause for why there is room for improvement in the measurement, even though there is no measurement error, can be acquired. Since the measurement temperature here is within the reference value for the temperature area, the (1) Temperature Area of the measurement variance cause information is recorded as “OK” as shown in FIG. 3A.

Next, the controller 5 checks the “(4) Deposited Amount” information (S415). More specifically, the improvement data acquisition component 13 determines whether or not there is an additional blood log in the memory 6, and if there is, it is recorded as “yes” that there is room for improvement in the measurement in the measurement improvement data 15. We will assume here that there is no additional blood log. As shown in FIG. 3A, in that case the (4) Deposited Amount of the measurement variance cause information is recorded as “OK.”

After this, the controller 5 displays the measured blood glucose value (80 in this case) on the display component 8.

The controller 5 also checks the “(A) Insertion/Removal” information or the “(B) Failure” information stored in the log as discussed above (S417). More specifically, the improvement data acquisition component 13 determines whether or not there is a sensor insertion/removal log in the memory 6. The fact that there is a sensor insertion/removal log indicates that the sensor has been inserted or removed between the last successful measurement and the current successful measurement. At this point, the improvement data acquisition component 13 records in the measurement improvement data 15 that there is room for improvement in the measurement. We will assume here that there is no sensor insertion/removal log. As shown in FIG. 3A, in this case the “(A) Insertion/Removal” of the measurement variance cause information is recorded as “OK.”

Also, the improvement data acquisition component 13 examines whether or not there is a measurement failure log in the memory 6. The fact that there is a measurement failure log indicates that measurement failed between the last successful measurement and the current successful measurement. Thus, the improvement data acquisition component 13 records to the measurement improvement data 15 that there is room for improvement in the measurement. We will assume here that there is no measurement failure log. As shown in FIG. 3A, in this case the “(B) Failure” of the measurement variance cause information is recorded as “OK.”

After this, the controller 5 deletes the sensor insertion/removal log and the measurement failure log (S418).

Finally, the controller 5 records the measurement improvement data 15 to the improvement data table 14 of the memory 6, and ends the measurement operation (S419).

As discussed above, when measurement is successful, the measurement improvement data 15 is associated with the blood glucose value for which measurement was successful in S413, and stored in the improvement data table 14 of the memory 6 in S419. As a result, the measurement improvement data 15 shown in FIG. 3A is completed. That is, in this embodiment, seven points of cause data (cause data 1 to 5 for measurement variance cause information and cause data A and B for measurement error cause information) are acquired with which the category to be improved can be determined for the usage state of the measurement device 1, and these are associated with measured values when measurement was successful and stored in the memory 6.

FIG. 3B shows the measurement improvement data 16, and was produced when the measurement performed after the measurement improvement data 15 was successful. This measurement improvement data 16 indicates data measured, for example, at 11:23 and 00 seconds on the morning of Nov. 12, 2012, and the blood glucose value at that time was 82. From this measurement improvement data 16 it can be understood that a state has occurred in which there should be improvement in the (1) Temperature Area, (2) Temperature Change, and (A) Insertion/Removal, which were all recorded as “yes.”

The controller 5 associates measured values with measurement improvement data every time a measurement is successful, and records the result in the memory 6. Consequently, the memory 6 stores a plurality of sets of measurement improvement data (such as the measurement improvement data 15 and the measurement improvement data 16). After this, the controller 5 outputs the measured value and the produced measurement improvement data 15 and measurement improvement data 16 through the communication component 11 to the outside. More specifically, as shown in FIG. 1, it is sent to the analyzer 17 (an example of a data processing device) of the physician (the person doing the analysis).

Data from the analyzer 17 is outputted to a display device. This display device displays the measurement error cause information and/or the measurement variance cause information held by the measurement improvement data 15 and the measurement improvement data 16. The display device in this embodiment is built into the main case of the analyzer 17 as the display component 18.

### 1-3 Analyzer 17

### 1-3-1 Configuration of Analyzer 17

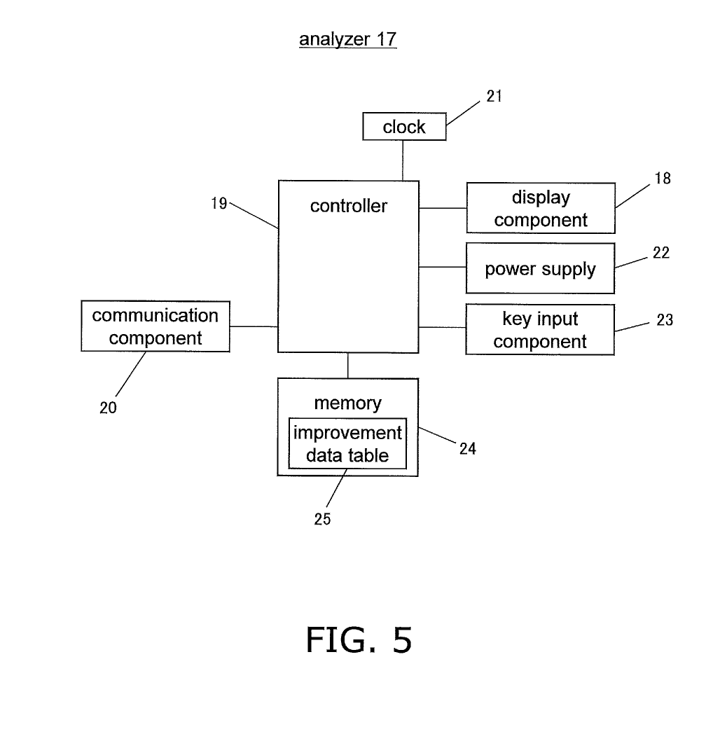

FIG. 5 shows the simplified configuration of the analyzer 17. The analyzer 17 comprises the display component 18, a controller 19, a communication component 20, a clock 21, a power supply 22, a key input component 23, and a memory 24. The display component 18 is constituted by an LCD, an organic EL display, or the like, and is electrically connected to the controller 19 inside the main case of the analyzer 17. The controller 19 is constituted by a CPU or other such processor that executes various functions according to specific programs. The controller 19 is connected to the communication component 20, the clock 21, the power supply 22, the key input component 23, and the memory 24.

When the measurement device 1 is connected to the analyzer 17 by a USB cable, for example, a plurality of sets of measured values and measurement improvement data associated with the measured values (such as the measurement improvement data 15 and the measurement improvement data 16 in FIGS. 3A and 3B) are transmitted from the communication component 11 of the measurement device 1.

### 1-3-2 Operation of Analyzer 17

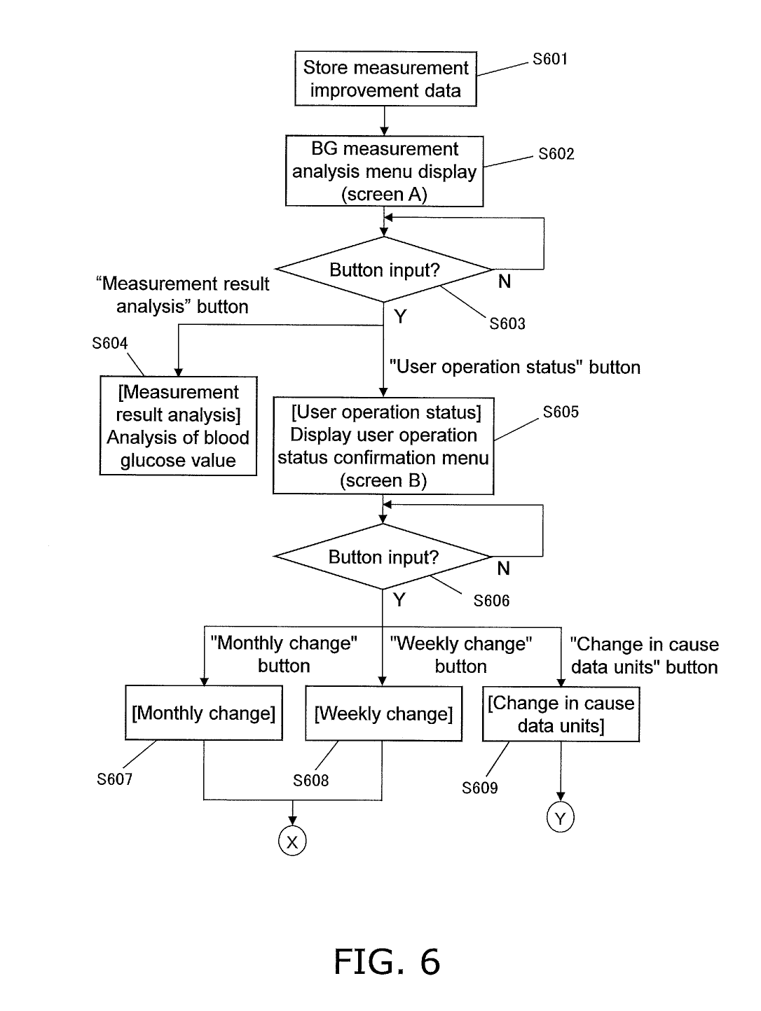

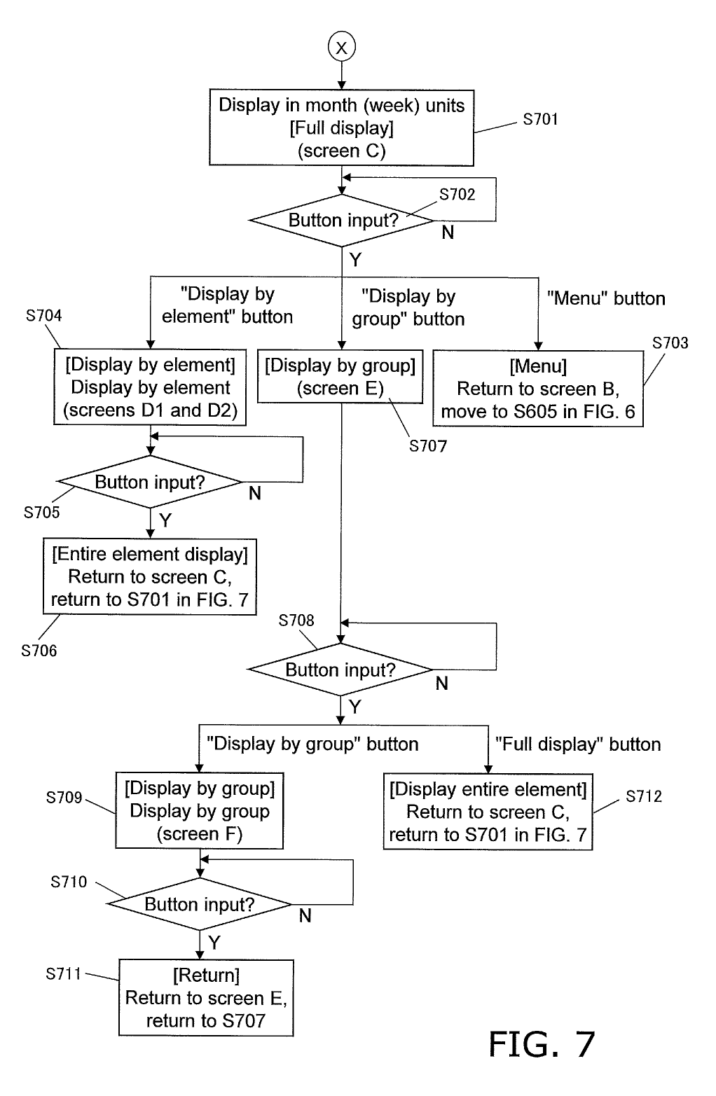

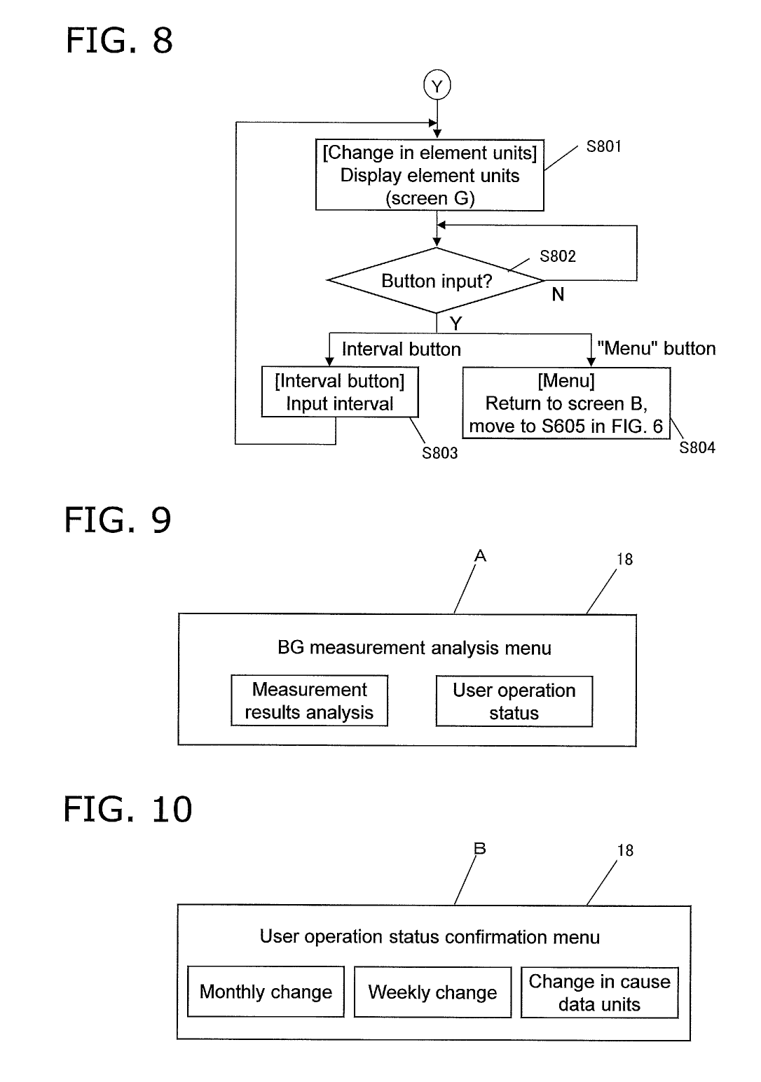

FIG. 6 shows the processing of measurement improvement data performed by the analyzer 17. When measurement improvement data transmitted from the measurement device 1 is received by the analyzer 17 via the communication component 20, the controller 19 of the analyzer 17 records the received measurement improvement data to an improvement data table 25 of the memory 24 (S601).

When the physician operates the key input component 23 of the analyzer 17, the controller 19 causes the display component 18 to display the screen A (BG measurement analysis menu) shown in FIG. 9 (S602). The system then awaits the button selection of the physician (the operator) (S603). “BG” in this embodiment stands for blood glucose, and indicates a blood glucose value.

Thereafter, the buttons displayed on the display component 18 are selected by the physician by operation of the key input component 23. Two buttons are displayed on the screen A: a “measurement result analysis” button and a “user operation status” button.

When the “measurement result analysis” button on screen A is selected, the controller 19 performs specific BG analysis (S604). This BG analysis is conventional analysis, and will not be described in detail here, but it involves management of the health of the user of the measurement device 1 on the basis of the trend in a plurality of blood glucose values sent along with measurement improvement data.

When the “user operation status” button on screen A is selected, as shown in FIG. 10, the controller 19 causes the display component 18 to display a “user operation status confirmation menu” on a screen B (S605). The system then awaits button operation by the physician (S606).

Thereafter, the physician uses the displayed screen to perform analysis related to measurement improvement.

Screen B displays three buttons: a “monthly change” button, a “weekly change” button, and a “change in cause data units” button.

When the “monthly change” button is selected, the controller 19 tallies the cause data (seven sets of cause data) constituting the measurement improvement data for that month, and displays the result on the display component 18. When the “weekly change” button is selected, the controller 19 tallies the cause data constituting the measurement improvement data for that week, and displays the result on the display component 18. When the “change in cause data units” button is selected, the controller 19 tallies the cause data constituting the measurement improvement data for each set of cause data, and displays the result on the display component 18.

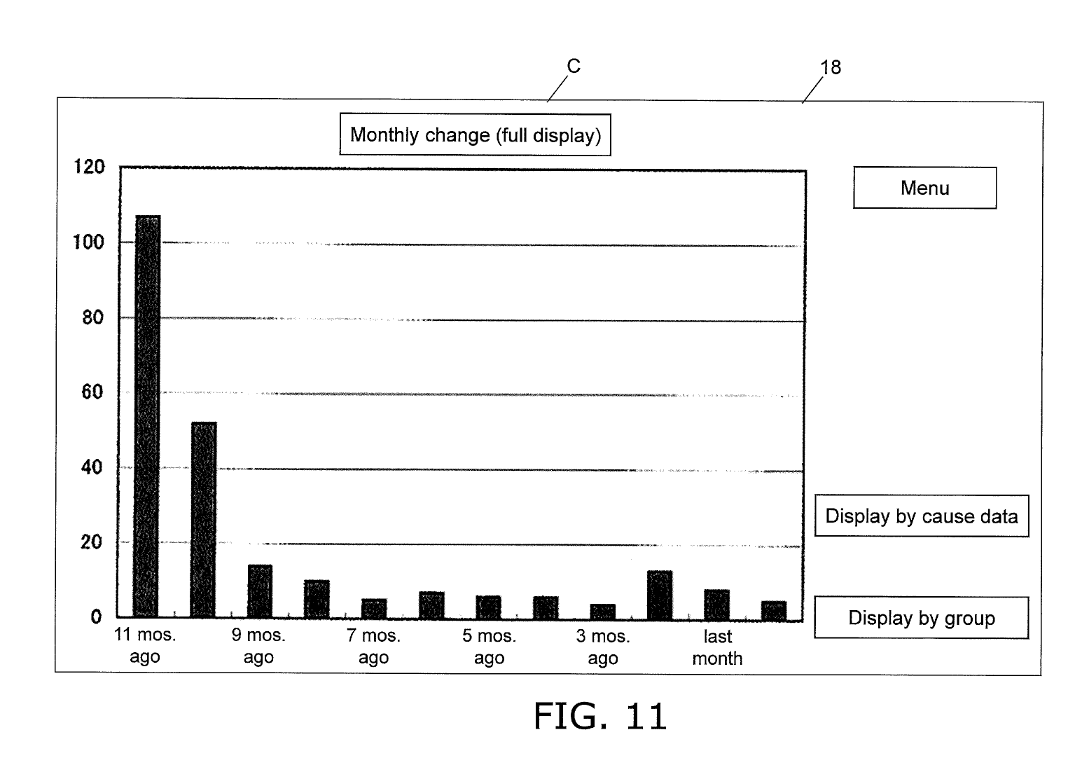

Specifically, when the “monthly change” button is selected on screen B (S607), the flow moves to the processing in FIG. 7, and the controller 19 displays a screen C (“monthly change” (full display)) on the display component 18 as shown in FIG. 11 (S701), and the system awaits button selection by the physician (S702).

On the screen C in FIG. 11, the controller 19 tallies the number of sets of cause data for that month in which there is room for improvement in the measurement, and displays a bar graph in time series. Since the number of sets of cause data in which there is room for improvement in the measurement is thus tallied on a monthly basis, the physician can ascertain the usage state of the measurement device 1, that is, the measurement skill of the patient in which there is room for improvement. As a result, the physician can advise the patient on the best way to use the measurement device 1. More specifically, the number of sets of cause data for which there is room for improvement in measurement by the user on screen C, for example, is 109 for 11 months ago, 52 for 10 months ago, nine for last month, and three for this month. That is, there is a declining trend in the number of sets of cause data in which there is room for improvement. Therefore, the physician can conclude that “this patient is learning to use the measurement device 1 properly,” and can advice the patient that he is “starting to be able to make measurements properly” on the basis of this analysis result.

There are three buttons displayed on the right side of the screen C: a “menu” button, a “display by cause data” button, and a “display by group” button. When the “menu” button is selected on screen C in FIG. 11, the controller 19 returns to S605 in FIG. 6 and causes the display component 18 to display the “user operation status confirmation menu” of screen B in FIG. 10 (S703).

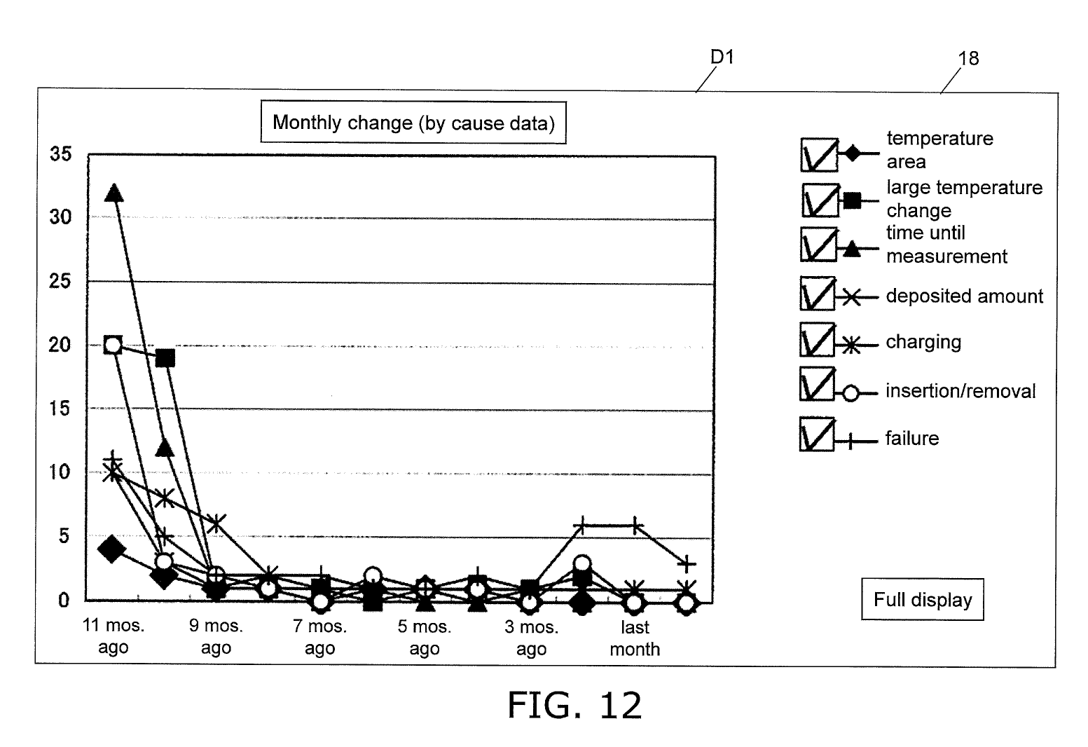

When the “display by cause data” button is selected on screen C in FIG. 11, the controller 19 causes the display component 18 to display a screen D1 (“monthly change” (by cause data)) as shown in FIG. 12 (S704 in FIG. 7), and the system awaits button selection by the physician (S705 in FIG. 7).

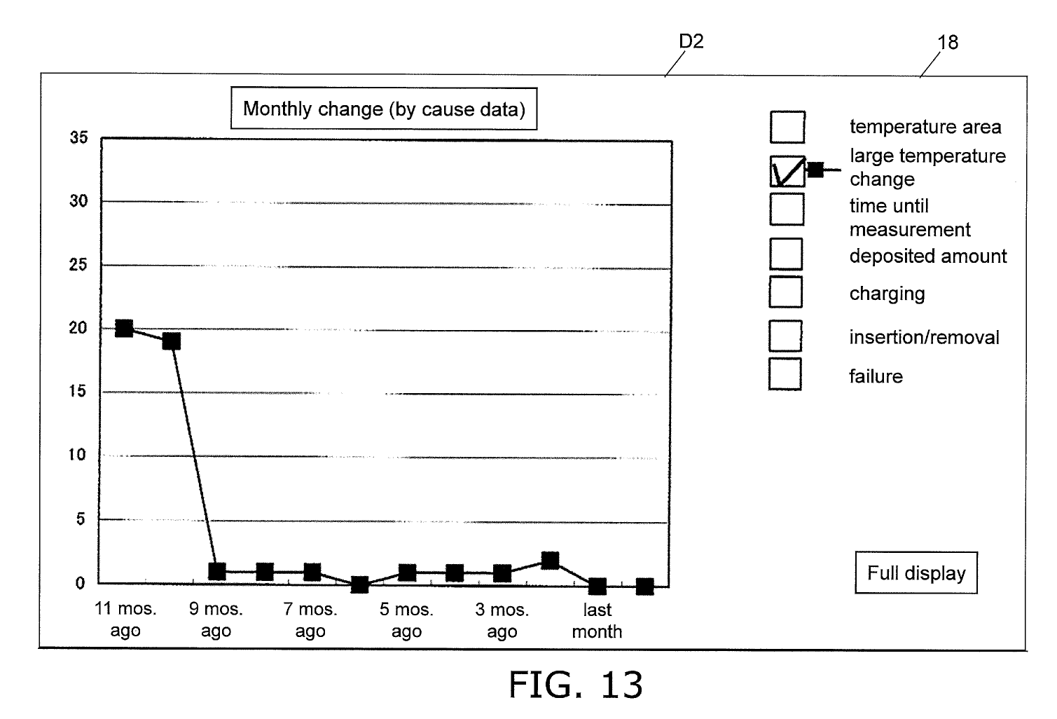

A checkbox for each cause data and a “full display” button are displayed on the right side of screen D1. On screen D1, the controller 19 tallies and displays cause data having room for improvement, on a monthly basis, for each cause data. This display can be displayed or not displayed for each cause data by checking or unchecking the checkboxes for cause data provided on the right side of the screen. For instance, as indicated by “monthly change (by cause data)” on screen D2 in FIG. 13, when just the checkbox for “large temperature change” is checked, the number of sets of cause data “temperature change” for which “yes” was recorded for room for improvement in the measurement is tallied on a monthly basis and displayed.

Returning to screen D1 in FIG. 12, to give an example of analysis, of the cause data for “(B) failure” (the + marks in FIG. 12), there are three this month, six last month, and six two months ago. Upon seeing this, the physician can conclude that “the number of measurement failures by this patient is high recently.” Based on this analysis result, the physician can advise the patient the best way to use the measurement device 1.

Examples of the advice the physician gives to the patient are given below for each cause data.

(1) Temperature area: “Take the measurement inside, or as close to 25° C. as possible.”

(2) Temperature change: “Don't measure while holding the device in your hands,” “Don't put the meter (measurement device 1) in your pocket, etc.,” “Use the meter after letting it adjust to its environment.”

(3) Time until measurement: “Once you insert a sensor, measure as soon as possible.”

(4) Deposited amount: “Be sure to deposit plenty of blood.”

(5) After charging: “Be sure to measure before charging.”

(A) Insertion/removal: “Don't keep inserting and removing the sensor.”

(B) Failure: “Follow the measurement instructions carefully.”

As a result, the user of the measurement device 1 can be encouraged to use the measurement device more properly.

When the “full display” button is selected on screen D1 in FIG. 12 (or on screen D2 in FIG. 13), the controller 19 causes the display component 18 to display screen C in FIG. 11 (monthly change (full display)) (S706), and the flow returns to S701.

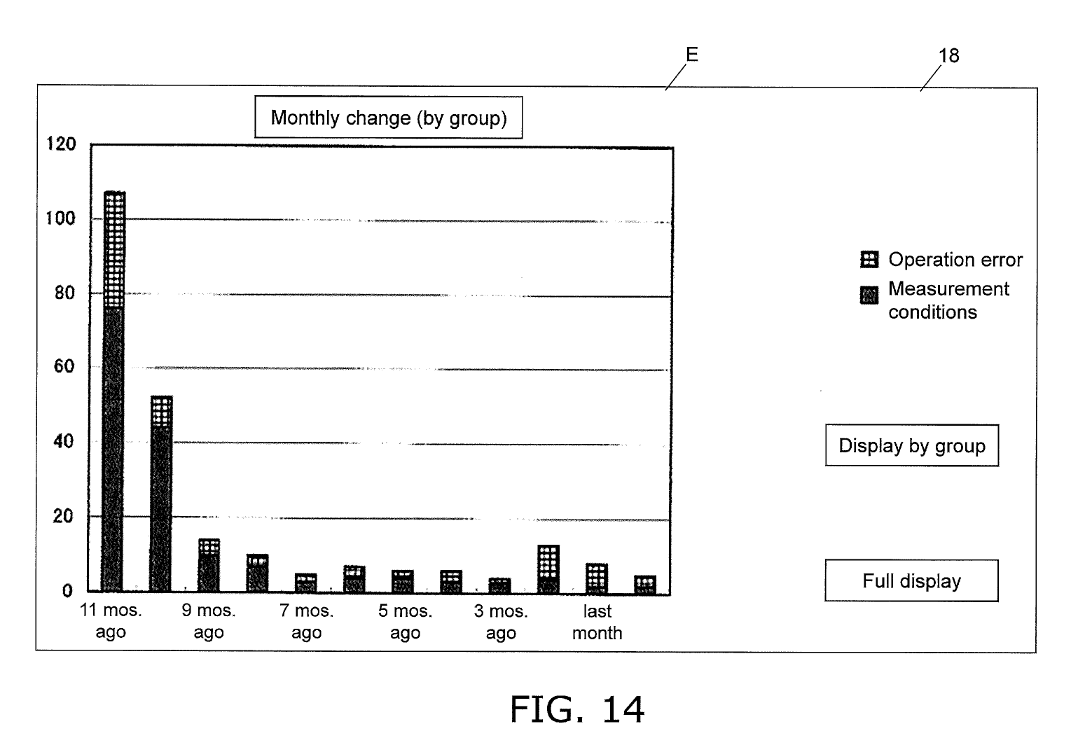

When the “display by group” button is selected on screen C in FIG. 11, the controller 19 causes the display component 18 to display screen E (monthly change (by group)) as shown in FIG. 14 (S707), and the system awaits button selection by the physician (S708).

Two buttons are displayed on the right side of screen E: a “display by group” button and a “full display” button. On screen E, the controller 19 puts the five sets of cause data constituting the measurement variance causes into a measurement condition group, and puts the two sets of cause data constituting the measurement error causes into a measurement error group, and tallies and displays the number of sets of cause data recorded as “yes” for room for improvement in the measurement, on a monthly basis, for each of these groups. For instance, the totals for the measurement condition group and the measurement error group are 109 for 11 months ago, 52 for 10 months ago, and nine for last month.

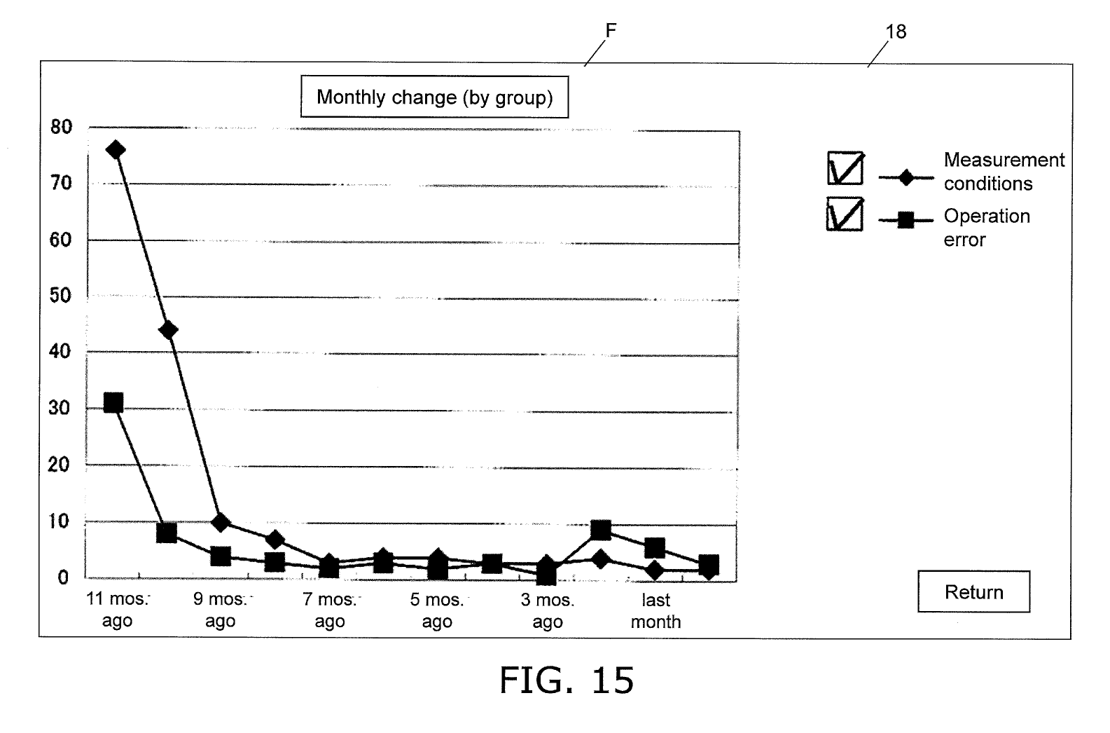

When the “display by group” button is selected on screen E, the controller 19 causes the display component 18 to display a screen F (monthly change (by group)) as shown in FIG. 15 (S709), and the system awaits button selection by the physician (S710).

A checkbox for each cause data group and a “return” button are displayed on the right side of screen F. In screen F, the controller 19 tallies and displays, on a monthly basis, improvement data for each group of cause data (the above-mentioned measurement condition group and the measurement error group). This display can be displayed or not displayed for each group by checking or unchecking the checkboxes for the groups provided on the right side of the screen.

As an example of analysis by the physician on screen F, the physician may conclude that “11 months ago, there were more measurement conditions (measurement variance causes) than measurement errors (measurement error causes), but in the past three months, there are more measurement errors (measurement error causes).” Upon reaching this conclusion, the physician can advise the patient on the best way to use the measurement device 1.

When the “return” button is selected on screen F, the controller 19 displays the screen E (monthly change (by group)) in FIG. 14 (S711), and the flow returns to S707.

When the “full display” button is selected on screen E (monthly change (by group)), the controller 19 displays screen C in FIG. 11 (S712), and the flow returns to S701.

When the “menu” button on screen C is selected, the flow returns to screen B in FIG. 10 (S605 in FIG. 6).

When the “weekly change” button on screen B is selected (S606 and S608 in FIG. 6), the controller 19 carries out the processing of the above-mentioned steps S701 to S12 in FIG. 7, but this time on a weekly basis. This will not be described here, to avoid complication.

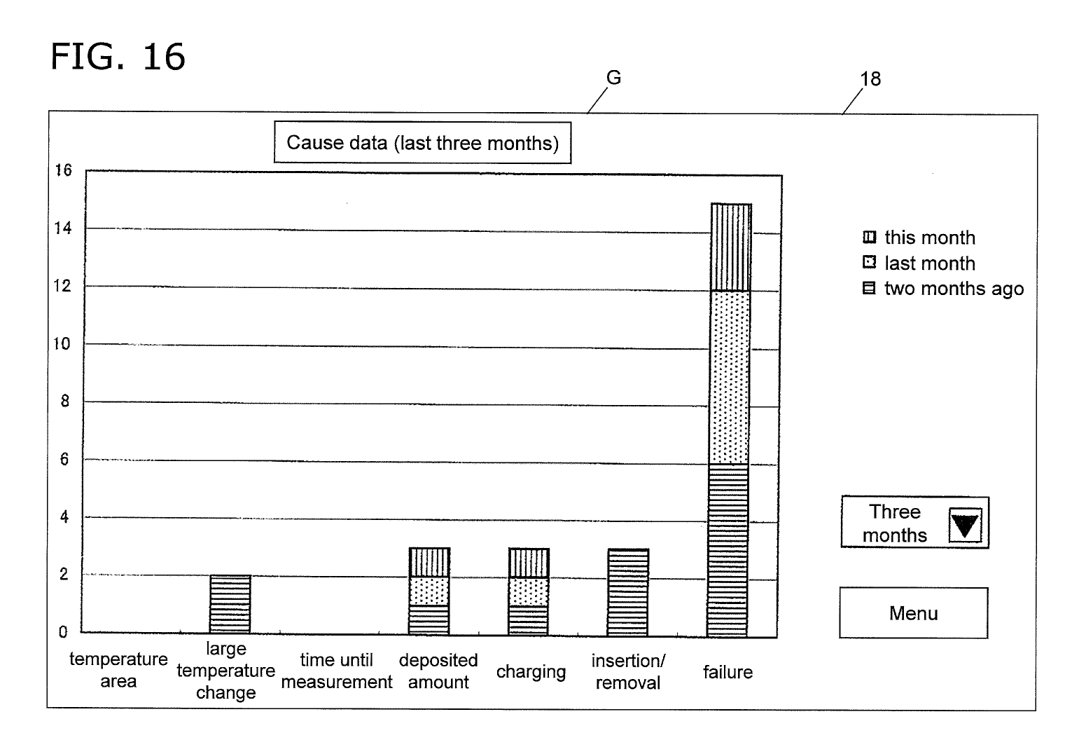

When the “change in cause data units” button is selected on screen B (S606 and S609 in FIG. 6), the controller 19 moves to the processing in FIG. 8, causes the display component 18 to display a screen G (cause data (last three months)) as shown in FIG. 16 (S8 in FIG. 8), and the system awaits button selection by the physician (S802).

Two buttons are displayed on the right side of screen G: a “three months” button (interval button), and a “menu” button. On screen G, the controller 19 tallies and displays the number of sets of cause data recorded as “yes” for room for improvement in the last three months, for example, for every cause data. This tally interval can be changed by the physician by selecting the “three months” button, and tally displays of one month or six months can be displayed instead, for example (S803).

To give an example of analysis by the physician on screen G, since there are many more instances of “failure” among the sets of cause data in the last three months, the physician concludes that “This patient often fails at measurement.” On the basis of this analysis, the physician can advise the patient the best way to use the measurement device 1.

When the “menu” button on screen G is selected (S804), the controller 19 returns to S605 in FIG. 6, displays screen B in FIG. 10, and awaits button selection by the physician.

The operation processing shown in FIGS. 6 to 9 is started by actuating a program, and is ended by ending that program.

### 1-4 Modification Example

In this embodiment, measurement variance cause information associated with measured values was sent to the analyzer 17 of the physician, allowing the physician to advise the patient on the best way to use the measurement device 1 on the basis of the measurement variance cause information displayed on the analyzer 17, but this is not the only option. Instead, the measurement variance cause information associated with measured values may be sent to a portable terminal belonging to the patient (an example of a data processing device; not shown). With this configuration, the measured values and the measurement variance cause information are displayed on the patient's portable terminal. Therefore, the patient himself can ascertain the usage state of the measurement device 1, and as a result, that patient will be encouraged to use the measurement device in a better way.

### 1-5 Effect, etc.

As described above, the measurement device 1 in this embodiment comprised the main case 1a that had the sensor mounting portion 3 to which the sensor 2 was mounted, the measurement component 4 that was connected to the sensor mounting portion 3, the controller 5 that was connected to the measurement component 4, the memory 6 that was connected to the controller 5, and the clock 7, and the controller 5 associated measurement variance cause information acquired by the controller 5 in the measurement of a blood glucose value with measured values obtained by the same measurement of blood glucose values, and stored this in the memory 6, so more favorable measurement could be performed with the measurement device 1.

Specifically, when measurement is executed, the controller 5 of the measurement device 1 encourages the patient to use the measurement device in a better way, acquires measurement variance cause information during measurement, associates this measurement variance cause information with the current measured value produced by the measurement component 4, and stores this in the memory.

Therefore, when the measured values stored in the memory 6 of the measurement device 1 are sent along with measurement variance cause information to the physician's computer, the physician can learn the usage state of the measurement device 1 on the basis of the measurement variance cause information displayed on the computer, and can advise the patient about the best way to use the measurement device 1. As a result, the user can be encouraged to use the measurement device 1 in a better way.

Also, if advice based on the measurement variance cause information stored in the memory 6 of the measurement device 1 is displayed on the display component 8 of the measurement device 1, the user himself will be able to understand the best way to use the measurement device 1.

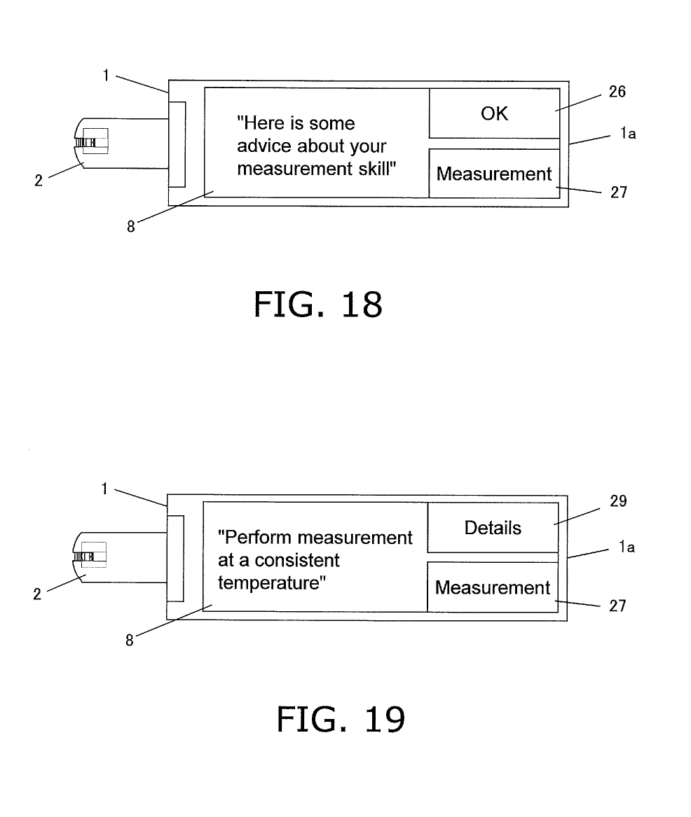

The operation of the measurement device 1 pertaining to Embodiment 2 will now be described mainly through reference to FIGS. 18 to 21.

In Embodiment 2, as shown in FIGS. 18 to 20, the configuration is such that when any set of measurement variance cause information is outside its reference value, measurement improvement advice is displayed on the display component 8 had by the measurement device 1 in Embodiment 1, prior to measurement, in order to improve the measurement skill of the user. The rest of the components and operation are the same as in Embodiment 1, and will be referred to using the same drawings and numbers. Redundant description of the components and operation may be omitted.

Again with the measurement device 1 pertaining to Embodiment 2, the user can be encouraged to use the measurement device better having the user himself check this measurement improvement advice.

The display component 8 of the measurement device 1 on which the measurement improvement advice is displayed is a touch screen, and the control keys are operated when the user touches buttons (an example of control keys) displayed on the screen, thereby causing the controller 5 to perform the control associated with those buttons. In this embodiment, buttons (an example of control keys) are displayed on the screen, but control keys may instead be provided to the main case 1a. Also, the measurement improvement advice in this embodiment is made up of three types of advice: advance notification to the effect that this advice will be given, a summary of the advice, and details of the advice. In this display, first the advance notification to the effect that advice will be given is displayed, then a summary of the advice is displayed, and after this the details of the advice are displayed.

The operation of the measurement device 1 will now be described by focusing on the differences from Embodiment 1.

### 2-1 Operation

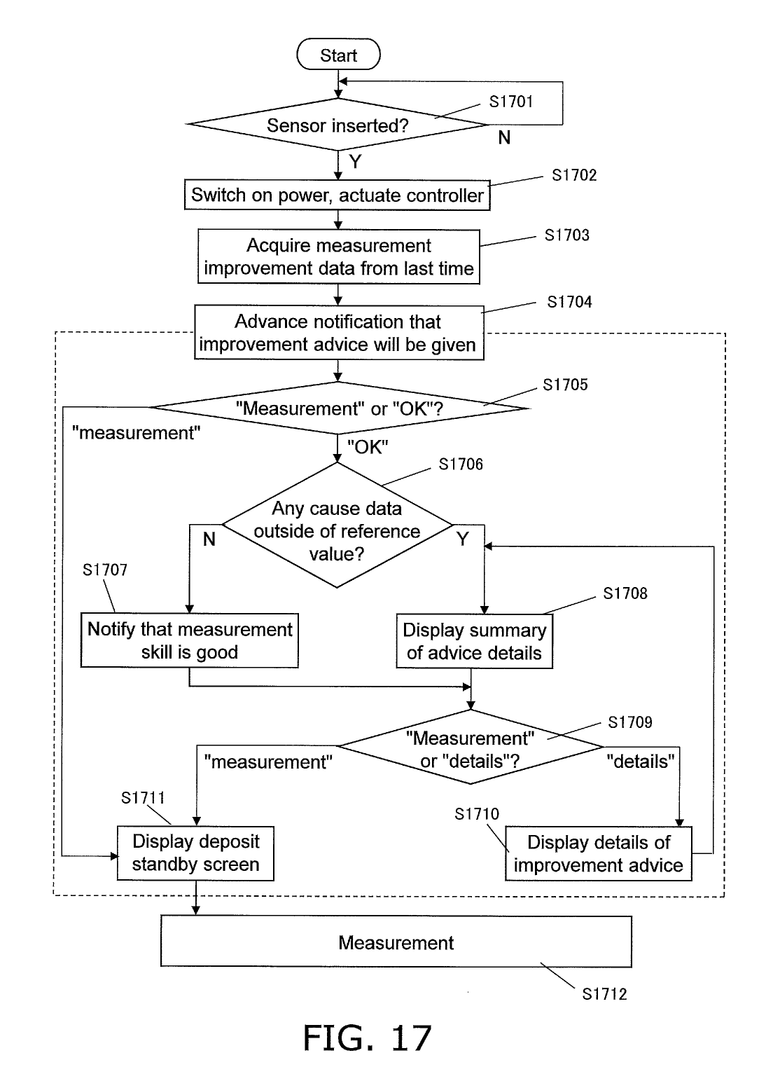

The operation of the measurement device 1 pertaining to this embodiment will now be described through reference to the flowchart in FIG. 17.

When the sensor 2 shown in FIG. 1 is mounted to the sensor mounting portion 3 shown in FIG. 2 (S1701), the power is switched on and the controller 5 of the measurement device 1 is actuated (S1702).

The controller 5 then acquires measurement improvement data for the last measurement from the improvement data table 14 of the memory 6 (S1703).

Then, as shown in FIG. 5, the controller 5 first displays “Here is some advice about your measurement skill,” for example, on the display component 8, thus giving the user advance notification that improvement advice about his measurement skill is about to be given (S1704). This display allows the user to find out that there is advice about his measurement skill before he performs the measurement.

Along with this advice notification, the controller 5 causes the display component 8 to display an OK button 26 (an example of a control key) and a measurement button 27 (an example of a control key) as shown in FIG. 18. The controller 5 determines which button has been operated (S1705).

When the user touches the measurement button 27, the flow proceeds to the processing of S1711, and shifts to the deposition of blood and the measurement of a blood glucose value.

When the user touches the OK button 26, the controller 5 determines whether or not any of the cause data recorded in the above-mentioned measurement improvement data (such as the measurement improvement data 15 in FIG. 3A and the measurement improvement data 16 in FIG. 3B) is outside its reference value. That is, it determines whether or not there is cause data outside its reference value (S1706).

If all seven of the cause data satisfy their reference values, the controller 5 refers to table data 28 (discussed below) and displays “The last measurement was good. Do it the same way this time” as a summary of the advice (S1707). This notification confirms to the user that there has been no problem with his measurement skill so far, and encourages him to continue using the measurement device 1 in the proper way.

On the other hand, if it is determined that there is cause data outside its reference value, the controller 5 refers to the table data 28 (discussed below) and displays a summary of the advice on the display component 8 (S1708).

The controller 5 in this embodiment causes the display component 8 to display a summary of the advice when any of the cause data (measurement variance cause information or measurement error cause information) is outside its reference value, if the user has touched the OK button 26 displayed on the display component 8 after being given advance notification that measurement improvement advice will be given. Accordingly, since a summary of the advice is displayed on the display component 8 of the measurement device 1 before measurement, the patient (the user) can perform the measurement after looking at this advice summary. As a result, the user can be encouraged to use the measurement device 1 in a better way.

A specific example will now be given. When there is cause data outside its reference value in the information about temperature during measurement, that is, (1) the temperature area or (2) the temperature change, the current advice summary is displays as “Perform measurement at a consistent temperature,” as shown in FIG. 19. Displaying such specific advice encourages the user to be careful to perform the current measurement at a consistent temperature. Also, since the advice summary is displayed after giving advance notification that this advice will be given, the user can properly understand what is being displayed.

In S1708, when the controller 5 causes the display component 8 to display a summary of improvement advice, a details button 29 and the measurement button 27 are displayed on the display component 8 as shown in FIG. 19. The controller 5 determines which of these buttons has been touched (S1709).

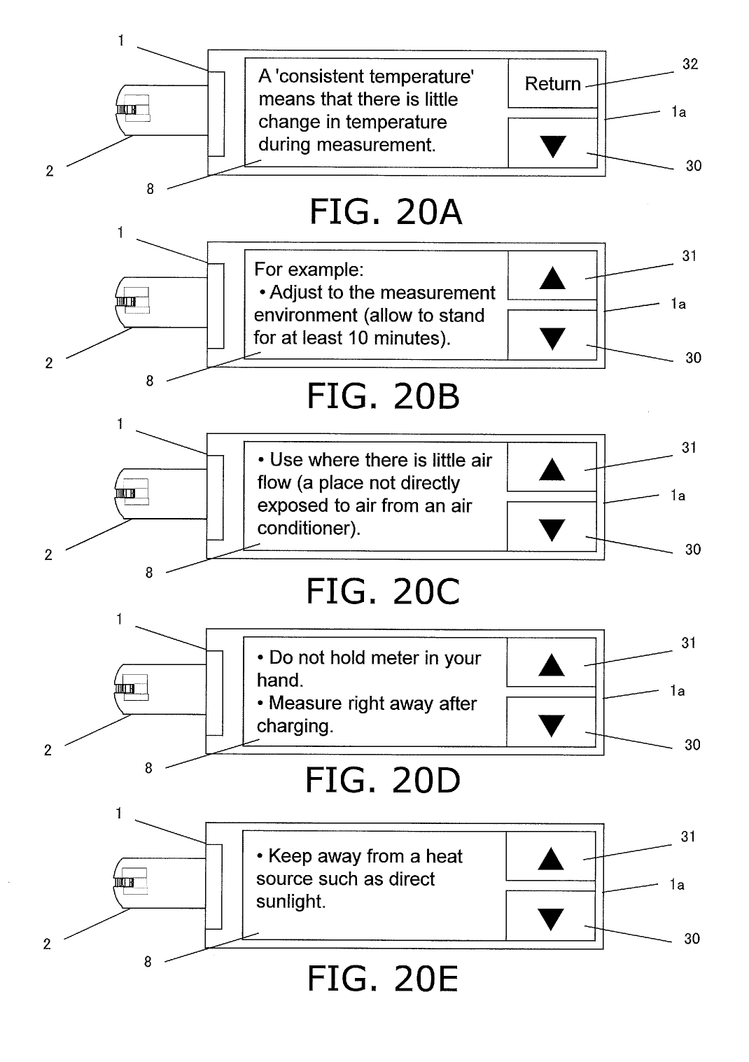

When the user touches the measurement button 27, the flow proceeds to S1711, and shifts to the deposition of blood and the measurement of a blood glucose value. On the other hand, when the user touches the details button 29, the controller 5 refers to the table data 28 (discussed below) and causes the display component 8 to display details about the improvement advice as shown in FIGS. 20A to 20E (S1710). The display of these details about improvement advice will be discussed below.

In S1710, if the user touches a return button 32 (an example of a control key) in FIG. 20A, the controller 5 returns to step S1708 in FIG. 17 and again displays a summary of advice as shown in FIG. 19 (S1708).

In S1705 or S1709, if the user touches the measurement button 27 (FIG. 18 or 19), the controller 5 causes the display component 8 to display a deposit standby screen (not shown), which prompts the user to deposit blood on the sensor 2 (S1711).

When blood is deposited on the sensor 2, the measurement component 4 performs measurement (S1712).

As discussed above, the controller 5 associates the current measured value obtained by the measurement component 4 with the measurement variance cause information fur the current measurement, and stores this in the improvement data table 14 of the memory 6.

After the sensor has been inserted in S1701, and the power has been switched on in S1702, the controller 5 may perform the processing of S403 to S409 in FIG. 4 in parallel with the processing of the above-mentioned S1703 to S1711. In this case, the flow shifts to the processing of S411 to S419 in FIG. 4 after the blood glucose value measurement in S1712.

### 2-2 Display of Advice Details

### 2-2-1 Advice Corresponding to Cause Data

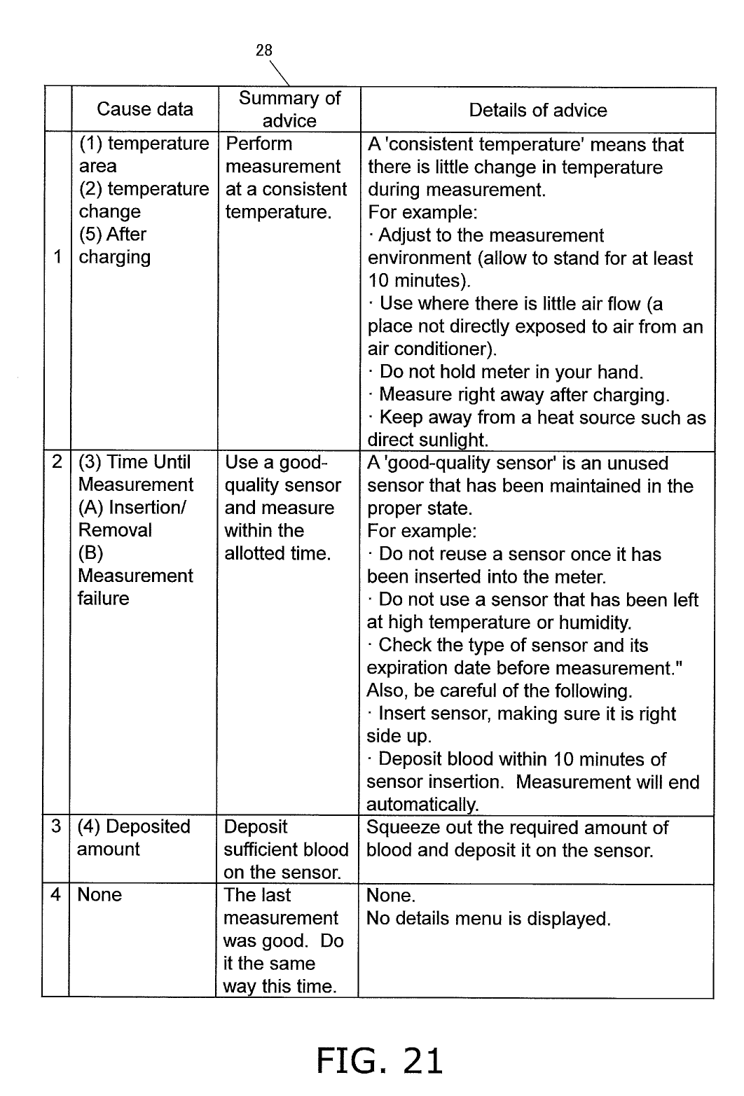

FIGS. 20a to 20e show examples of the display of advice details based on measurement improvement data. FIG. 21 shows the table data 28, which lists the relation between cause data, advice summary, and advice details. This table data 28 is stored ahead of time in the memory 6. The configuration of this table data 28 will be described below.

As shown in FIG. 21, the table data 28 is divided into four groups, by cause data, and includes advice summaries and advice details for each cause data.

In the first group, the cause data are (1) temperature area and (2) temperature change, which are information about the temperature during measurement, and (5) after charging, which is information about the length of time since charging. If cause data in the first group is outside its reference value, the summary of advice will be, for example, “Perform measurement at a consistent temperature.”

The details of advice for the first group include the following five, for example.

1. “A ‘consistent temperature’ means that there is little change in temperature during measurement.”

2. “For example: •Adjust to the measurement environment (allow to stand for at least 10 minutes).”

3. “•Use where there is little air flow (a place not directly exposed to air from an air conditioner).”

4. “•Do not hold meter in your hand. •Measure right away after charging.”

5. “•Keep away from a heat source such as direct sunlight.”

In the second group, the cause data is (3) time until measurement, which is information about the time between the mounting of the sensor 2 to the sensor mounting portion 3 and measurement by the measurement component 4, and (A) insertion/removal and (B) failure, which are measurement error cause information. If there is cause data in the second group that is outside its reference value, the summary of advice is, for example, “Use a good-quality sensor and measure within the allotted time.”

The details of advice for the second group include the following six, for example.

1. “A ‘good-quality sensor’ is an unused sensor that has been maintained in the proper state.”

2. “For example: •Do not reuse a sensor once it has been inserted into the meter.”

3. “Do not use a sensor that has been left at high temperature or humidity.”

4. “Check the type of sensor and its expiration date before measurement.”

5. “Be careful of the following. •Insert sensor, making sure it is right side up.”

6. “Deposit blood within 10 minutes of sensor insertion. Measurement will end automatically.”

In the third group, the cause data is (4) deposited amount, which is information about the amount of blood deposited on the sensor 2. If cause data in the third group is outside its reference value, the summary of advice will be, for example, “Deposit sufficient blood on the sensor.”

The details of advice for the fourth group includes the following one, for example.

1. “Squeeze out the required amount of blood and deposit it on the sensor.”

The fourth group is advice information for when there is no cause data outside its reference value. The advice summary is, for example, “The last measurement was good. Do it the same way this time.” Also, in this case, there is no data for advice details, and no details menu is displayed.

### 2-2-2 Example of Display of Advice Details

As discussed above, the controller 5 in this embodiment determines whether or not there is cause data outside its reference value, refers to the table data 28, and causes the display component 8 to display an advice summary and advice details.

Next, the display of advice details will be described.

As shown in FIG. 19, when an advice summary is displayed, the controller 5 causes the display component 8 to display the details button 29 (an example of a control key) and the measurement button 27. After the advice summary has been displayed, if the user touches the details button 29, details about the measurement improvement advice are displayed on the display component 8 (S1710 in FIG. 17).

To give a specific example, if any of the following cause data belonging to the first group of the table data 28 in FIG. 21, namely, (1) temperature area, (2) temperature change, and (5) after charging, is outside its reference value, then the controller 5 takes the advice details corresponding to that cause data from the table data 28 in FIG. 21, and causes the display component 8 to successively display the details data as shown in FIGS. 20a to 20e.

The details of the advice are switched by the user by touching a next button 30 (an example of a control key) or a previous button 31 (an example of a control key) displayed on the display component 8. This time, as shown in FIGS. 20a to 20e, the following five sets of advice details are successively switched and displayed.

1. “A ‘consistent temperature’ means that there is little change in temperature during measurement.” (FIG. 20A)

2. “For example: •Adjust to the measurement environment (allow to stand for at least 10 minutes).” (FIG. 20B)

3. “•Use where there is little air flow (a place not directly exposed to air from an air conditioner).” (FIG. 20C)

4. “•Do not hold meter in your hand. •Measure right away after charging.” (FIG. 20D)

5. “•Keep away from a heat source such as direct sunlight.”

### 2-3 Modification Example

With the above-mentioned measurement device 1, the measurement improvement data the controller 5 acquires from the improvement data table 14 of the memory 6 is not limited to that from the current measurement. When a plurality of measurements are made after finally giving improvement advice, the plurality of sets of measurement improvement data acquired during these measurements may be stored ahead of time in the memory 6, and the above-mentioned improvement advice conveyed on the basis of the plurality of sets of measurement improvement data.

### 2-4 Effect, etc.

As described above, the controller 5 in Embodiment 2 is configured to cause the display component 8 to display measurement improvement data prior to measurement by the measurement component 4 if there is any cause data (measurement variance cause information or measurement error cause information) outside of its reference value. Therefore, the user can be encouraged to use the measurement device in a better way.

Specifically, if there is any cause data (measurement variance cause information or measurement error cause information) outside of its reference value, the controller 5 causes the display component 8 to display measurement improvement data based on the cause data stored in the memory of the measurement device, prior to measurement by the measurement component 4. Accordingly, the user can be given advice about his measurement skill prior to measurement. Therefore, the person taking the measurement can himself understand the best way to use the measurement device 1. As a result, the user can be encouraged to use the measurement device in a better way.

### Other Embodiments

Embodiments were described above as examples of the technology disclosed herein, but the technology disclosed herein is not limited to or by these, and can also be applied to embodiments with modifications, substitutions, additions, omissions, and so forth made as needed. Also, the various constituent elements described in the embodiments above can be combined to create new embodiments.

In view of this, some other embodiments are given below.

[1]

Notification between the measurement device 1 and the analyzer 17 may be either wired or wireless. Also, the data including the measurement improvement data of the measurement device 1 may be transmitted to a PC, a portable terminal, or another such communications device connected to the measurement device 1, and may be transmitted from this communications device through a network to the analyzer 17.

Also, when the user utilizes a communications device connected to the measurement device 1, data from the communications device may be transmitted to and stored in a server device. In this case, the server device transmits measurement improvement data at a request from the analyzer 17. Also, in this case, the controller of the server device may take on some or all of the functions of the controller 19 of the analyzer 17 (recording of the measurement improvement data and analysis of the measurement improvement data).

[2]

In the above embodiments, the controller 5 of the measurement device 1 acquired measured values and the date and time of measurement as measurement results in measurement improvement data, but this is not the only option. The measurement results may be just the date and time of the measurement, or just the measurement date, and this data may be associated with measurement variance cause information or other such cause data before being transmitted to a data processing device.

[3]

The screen layout, display mode, advice content, button display, and so forth outputted to the display component 8 of the measurement device 1 and the display component 18 of the analyzer 17 are just examples, and are not limited to what was given above.

[4]

In the above embodiments, a blood glucose value measurement device was given as an example of the measurement device 1, but this is not the only option, and any device may be used as long as it measures biological information (cholesterol level, neutral fat level, albumin level, globulin level, oxygen saturation, hemoglobin level, myoglobin level, uric acid level, etc.) using a substance obtained from an organism (such as blood, urine, tissue, or cells) as a biological sample.

[5]

The execution order in the processing method given in the above embodiments is not necessarily limited to what was given in the above embodiments, and can be switched around to the extent that this does not depart from the gist of the invention.

[6]

Embodiments of the present invention are not limited to being worked as a biological information measuring device or system as in the above embodiments, and can also be worked as a method for encouraging the proper use of a biological information measuring device.

### INDUSTRIAL APPLICABILITY

Embodiments of the present invention are expected to find wide application as a biological information measuring device and as a biological information measurement system in which this device is used.

1. A biological information measuring device, comprising:
a main case;
a sensor mounting portion that is provided near the main case and is used to mount a sensor for measuring biological information;
a measurement component that is connected to the sensor mounting portion;
a controller that is connected to the measurement component;
a memory that is connected to the controller; and
wherein the controller, in a single measurement of biological information by the measurement component, acquires measurement variance cause information which causes an occurrence of measurement variance within a range of permissible error in a measured value acquired by the single measurement of biological information,
acquires each of a respective reference value for the measurement variance cause information,
produces measurement improvement data including a result of determining whether or not the measurement variance cause information is outside reference values, and
associates the measurement improvement data with measurement results obtained by the single measurement of biological information when the single measurement is of biological information successful, and stores the measurement improvement data in the memory.

2. The biological information measuring device according to claim 1, further comprising:
a timer,
wherein the measurement variance cause information includes one or more of information about a duration from when the sensor is mounted to the sensor mounting portion until the measurement component measures the biological information using the timer, and information about a quantity of biological sample deposited on the sensor.

3. (canceled)

4. The biological information measuring device according to claim 1,
wherein the controller acquires measurement error cause information which includes one or more of sensor insertion/removal information indicating whether or not the sensor has been inserted into or removed from the sensor mounting portion, and information about measurement failure at the measurement component in the measurement of the biological information; and
associates the measurement error cause information with the measurement results obtained in the single measurement of biological information, and stores the measurement error cause information in the memory when the single measurement of biological information is successful.

5. The biological information measuring device according to claim 4,
wherein the controller further:
acquires each of a reference value for the measurement error cause information;
produces measurement improvement data including the result of determining whether or not the measurement error cause information is outside the reference values; and
associates the measurement improvement data with the measurement results and stores the measurement improvement data in the memory.

6. The biological information measuring device according to claim 1,
wherein the controller stores the measurement improvement data in the memory every time the biological information is measured, and
the memory stores a plurality of sets of measurement improvement data.

7. The biological information measuring device according to claim 6,
further comprising a communication component capable of communicating with an external device,
wherein the controller outputs the measurement improvement data associated with the measurement results through the communication component to the external device.

8. The biological information measuring device according to claim 1
further comprising a display component,
wherein, when there is at least one piece of measurement variance cause information determined to be outside the reference values, the controller displays prompting the user to improve the measurement technique, on the display component prior to measurement of the biological sample by the measurement component.

9. The biological information measuring device according to claim 8,
wherein the controller displays on the display component an advance notification that the improvement advice based on the measurement variance cause information stored in the memory will be given.

10. The biological information measuring device according to claim 9,
wherein the controller displays on the display component a summary of the improvement advice after the advance notification.

11. The biological information measuring device according to claim 10,
wherein the summary of the improvement advice is advice about one or more of information about the duration from when the sensor is mounted to the sensor mounting portion until the measurement component measures the biological information, and information about the quantity of biological sample deposited on the sensor.

12. The biological information measuring device according to claim 11,
wherein the controller displays on the display component details about the improvement advice after the summary of the improvement advice has been displayed on the display component.

13. The biological information measuring device according to claim 12,
wherein the details of the improvement advice include advice about one or more of the pieces of information about the duration from when the sensor is mounted to the sensor mounting portion until the measurement component measures the biological information, and information about the quantity of biological sample deposited on the sensor.

14. The biological information measuring device according to claim 2,
further comprising a rechargeable battery,
wherein the measurement variance cause information includes time information indicating an amount of time from a completion of the charging of the rechargeable battery until the biological information measuring device is actuated.

15. A biological information measurement system, comprising:
logical information measuring device according to claim 7;
a data processing device that receives measurement improvement data associated with the measurement results from the communication component of the biological information measuring device; and
a display device that is connected to the data processing device,
wherein the data processing device outputs to the display device information about the measurement variance cause information and/or the measurement error cause information, out of the measurement improvement data.

16. The biological information measurement system according to claim 15,
wherein the data processing device outputs to the display device one or more sets of the measurement improvement data.

17. The biological information measurement system according to claim 16,
wherein the data processing device tallies the plurality of sets of measurement improvement data at specific intervals, and outputs a tally result to the display device.

18. A method in which a biological information measuring device is used for measuring biological information with a sensor, the method comprising the steps of:
acquiring, during the measurement of the biological information, measurement variance cause information which causes the occurrence of measurement variance within a range of permissible error in a measured value acquired by the measurement of the biological information, measurement variance cause information that includes one or more pieces of information about the duration from when the sensor is mounted to the biological information measuring device until the sensor measures the biological information, and information about a quantity of biological sample deposited on the sensor;
producing measurement improvement data indicating whether or not the measurement variance cause information is outside reference values every time the biological information is measured;
associating the produced measurement improvement data with measurement results obtained by the measurement of the biological information when the measurement of the biological information is successful and storing the produced measurement improvement data in a memory; and
outputting an analysis result based on the measurement improvement data stored in the memory to a display component.

19. A biological information measuring device, comprising:
a main case;
a sensor mounting portion that is provided near the main case and is used to mount a sensor for measuring biological information;
a measurement component that is connected to the sensor mounting portion;
a controller that is connected to the measurement component;
a memory that is connected to the controller; and
a timer;
wherein the controller, in a single measurement of biological information by the measurement component, acquires measurement variance cause information which causes an occurrence of measurement variance within a range of permissible error in a measured value acquired by the single measurement of biological information, measurement variance cause information that includes one or more of pieces information about a duration from when the sensor is mounted to the biological information measuring device until the sensor measures the biological information using the timer, and information about a quantity of biological sample deposited on the sensor;
associates the measurement variance cause information with the measurement results obtained by the single measurement of biological information when the single measurement of biological information is successful, and stores the measurement variance cause information in the memory.
